(19) United States
(12) Patent Application Publication
(10) Pub. No.: US 2003/0204262 A1
(43) Pub. Date: Oct. 30, 2003

(54) Quick disconnect orthopaedic trials

(76) Inventors: Joe William Ferguson, Issaquah, WA (US); James Ray Rogers, Winona Lake, IN (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/135,610

(22) Filed: Apr. 30, 2002

(51) Int. Cl. A61F 2/30; A61B 17/16; A61F 2/46

(52) U.S. Cl. 623/20.15; 606/80; 606/84; 623/23.44; 623/22.41; 623/22.42

(57) ABSTRACT

Modular orthopaedic components are disclosed. One set comprises orthopaedic trials and the other set comprises orthopaedic instruments. Both sets provide several interchangeable elements. Two or more modular components can be quickly and easily assembled and disassembled. Some of the modular components have open female ends with interior grooves that hold a garter-type canted coil spring. Others of the modular components have male ends with exterior grooves. When the two ends are pushed together, the canted coil spring is received in the aligned grooves, temporarily holding the elements together until the surgeon uses force to pull them apart. No special tool is needed to assemble and disassemble these modular components.

### FIELD OF THE INVENTION

The present invention relates to orthopaedic trials that can be quickly connected and disconnected during orthopaedic surgery.

### BACKGROUND OF THE INVENTION

Some orthopaedic surgical procedures are performed after severe bone loss due to disease or trauma. In the leg, bone loss can, for example, extend for a portion of the length of the femur, or some conditions may require replacement of an entire femur. Other bone portions can also be surgically removed due to disease or trauma. For example, the entire knee joint can be removed, along with portions of the patient's tibia.

For treatment of such severe bone losses, it is desirable to utilize a system of implants that allow some flexibility to the surgeon. For example, it is desirable that the surgeon have options in the length of the implant to be used, to accommodate the patient's unique needs.

During such surgical procedures, orthopaedic trials are used to assist a surgeon in preparing the bones for implantation of artificial joint parts. A surgeon uses a trial to ensure that the proper implant size will be used, to make the appropriate cuts and reams in the bone, and to ensure a proper alignment and component thickness prior to implanting the components.

For the orthopaedic trials to be most useful, it is desirable that they mimic the sizes and shapes of the final implant components to be used. Therefore, it is desirable that the orthopaedic trials offer the same flexibility as offered by the final implants. To optimize the utility of such orthopaedic trials, it is desirable that these orthopaedic trials also be easily and quickly assembled or connected and disassembled or disconnected.

### SUMMARY OF THE INVENTION

The present invention addresses the need for orthopaedic trials that offer flexibility in size and shape, and that can be easily and quickly assembled or connected and disassembled and disconnected.

In one aspect, the present invention provides a set of modular orthopaedic components comprising a first orthopaedic component having an interior groove, a second orthopaedic component having an exterior groove and a garter-type canted coil spring received in one of the grooves.

In another aspect, the present invention provides an assembly of modular orthopaedic components comprising a first modular orthopaedic element having a bore and an interior groove, a second modular orthopaedic element having a male portion with an exterior groove, and a garter-type canted coil spring. The male portion of the second modular orthopaedic element is received in the bore of the first modular orthopaedic element and at least part of the garter-type canted coil spring is received within the interior groove of the first modular orthopaedic element and at least part of the garter-type canted coil spring is received with the exterior groove of the second modular orthopaedic element.

In either case, the orthopaedic elements or components may be modular trials, such as modular femoral and tibial trials, or modular instruments, such as modular reamers and planars.

### BRIEF DESCRIPTION OF THE DRAWINGS

In the accompanying drawings, like reference numbers are used for like parts, and:

FIG. 1 is a perspective view of a distal femoral trial employing the principles of the present invention;

FIG. 2 is an elevation of the distal femoral trial of FIG. 1, with part shown in cross-section;

FIG. 3 is a cross-section of the distal femoral trial of FIGS. 1-2, taken along line 3-3 of FIG. 2;

FIG. 4 is an elevation of a tibial trial employing the principles of the present invention;

FIG. 5 is a top plan view of the tibial trial of FIG. 4;

FIG. 6 is a cross-section of the tibial trial of FIGS. 4-5, taken along line 6-6 of FIG.5;

FIG. 7 is an elevation of a first embodiment of a segmental component trial employing the principles of the present invention;

FIG. 8 is a cross-section of the segmental component trial of FIG. 7, taken along line 8-8 of FIG. 7;

FIG. 9 is an elevation of a second length of segmental component trial employing the principles of the present invention;

FIG. 10 is an elevation another embodiment of a segmental component trial employing the principles of the present invention, the segmental component trial having two male ends;

FIG. 11 is a cross-section of another embodiment of a segmental component trial employing the principles of the present invention, the segmental component trial having two female ends;

FIG. 12 is an elevation of a stem trial employing the principles of the present invention;

FIG. 13 is an elevation of a proximal femoral trial employing the principles of the present invention;

FIG. 14 is a cross-section of the proximal femoral trial of FIG. 13;

FIG. 15 is a top plan view of a garter-type canted coil spring;

FIG. 16 is a cross-section of the garter-type canted coil spring of FIG. 15, taken along line 16-16 of FIG. 15;

FIG. 17 is a cross-section of the modular segmental component trial of FIGS. 7-8, shown assembled with a canted-coil spring;

FIG. 18 is a cross-section of two modular trials, shown assembled together;

FIG. 19 is an elevation of a reamer with a male end incorporating the principles of the present invention;

FIG. 20 is an elevation of a Calcar Planar employing the principles of the present invention; and

FIG. 21 is a cross section of one end of the Calcar Planar of FIG. 20.

### DETAILED DESCRIPTION

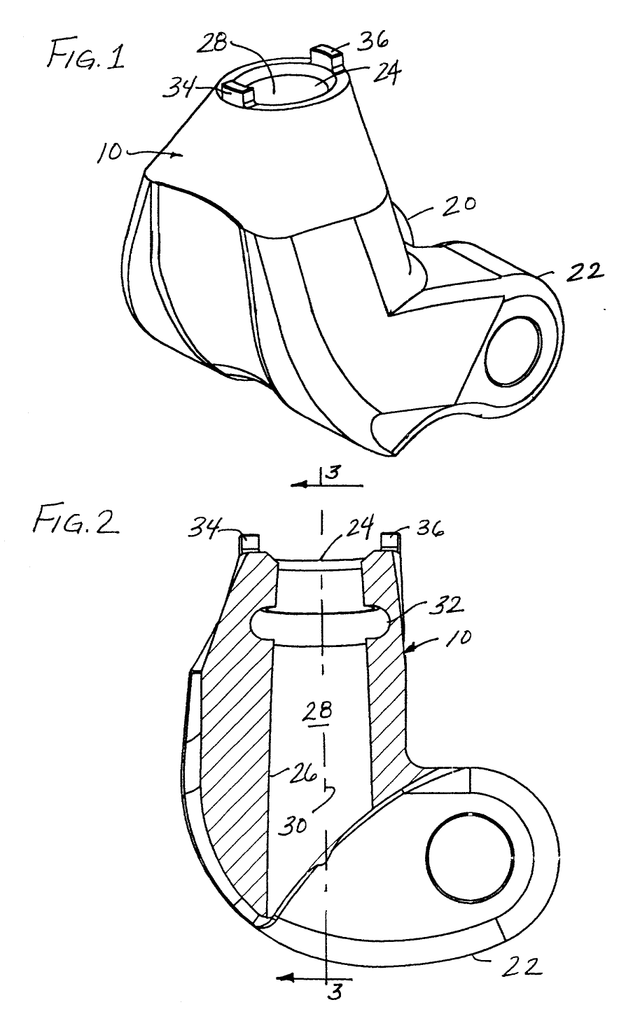

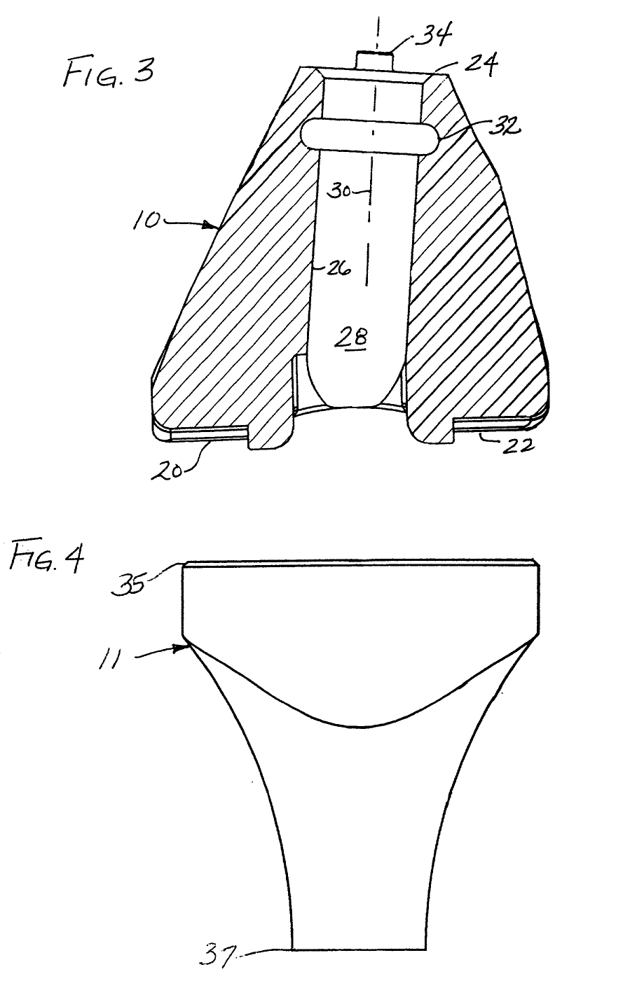

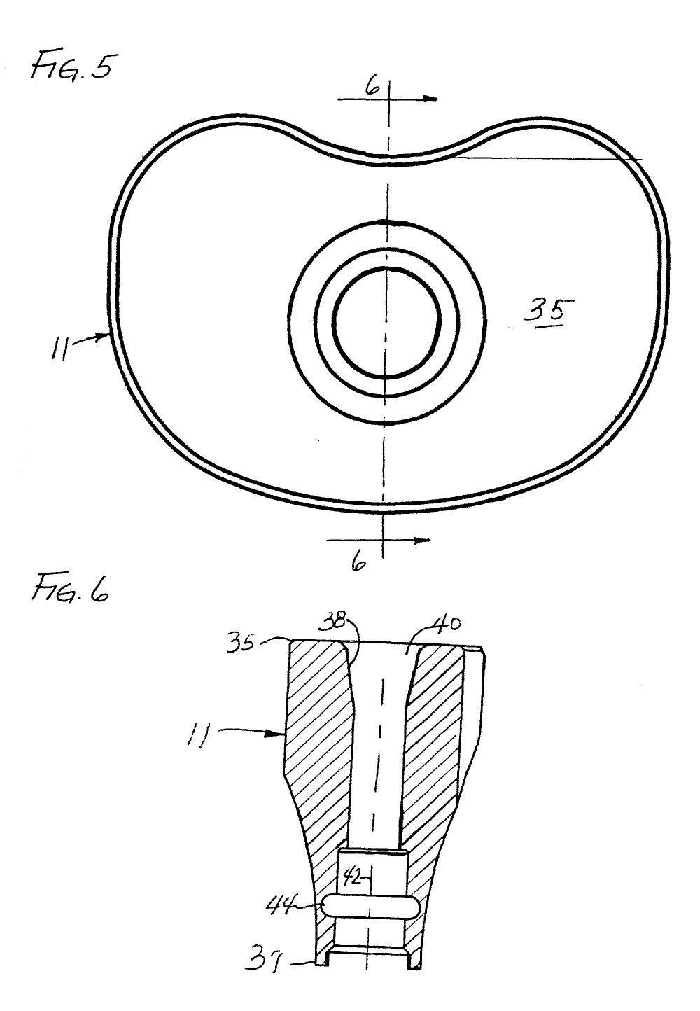

A set of modular orthopaedic trials utilizing the principles of the present invention are illustrated in the accompanying drawings, FIGS. 1-11. The illustrated set includes several elements: first through eighth orthopaedic trials 10, 11, 12, 12A, 13, 14, 15, 16. It should be understood that the particular orthopaedic trials shown and the number of orthopaedic trials shown are not intended to limit the invention in any way unless expressly called for in the claims. It is expected that actual embodiments of the invention will include more trials than those shown.

The illustrated modular orthopaedic trials 10, 11, 12, 12A, 13, 14, 15, 16 are for use in replacing portions of a patient's leg bones, including at least parts of the femur and tibia. However, it should be understood that the principles of the present invention are believed to be applicable to other bones and joints, and the invention is not intended to be limited to the bones of the leg unless expressly called for in the claims.

An embodiment of a first orthopaedic trial 10 is illustrated in FIGS. 1-3. The illustrated first orthopaedic trial 10 comprises a distal femoral component trial. It's exterior shape is similar to that of the modular distal femoral implant component that will ultimately be used, with two condylar portions 20, 22 and an open proximal end 24 opposite the condylar portions 20, 22. The first orthopaedic trial 10 includes an interior wall 26 that defines a bore 28 with a longitudinal axis 30. The interior wall 26 also has a circular interior groove 32 near the proximal end 24. In the illustrated embodiment, the interior groove 32 is spaced 0.326 inches from the open proximal end 24; the illustrated interior groove 32 comprises a full radius of 0.200 inches. The illustrated first orthopaedic trial also has a pair of upstanding anti-rotation tabs 34, 36. It should be understood that the dimensions and particular shape and features of the illustrated first orthopaedic component are provided for purposes of illustration only; the invention is not intended to be limited to these dimensions, shape or features unless expressly called for in the claims.

An embodiment of a second orthopaedic trial 11 is illustrated in FIGS. 4-6. The illustrated second orthopaedic trial 11 comprises a proximal tibial replacement trial. It's exterior shape is similar to that of the modular proximal tibial implant component that will ultimately by used, with proximal and distal ends 35, 37 respectively. As shown in FIG. 6, the second orthopaedic trial 11 includes an interior wall 38 that defines a bore 40 with a longitudinal axis 42. The interior wall 38 also has a circular interior groove 44 near the distal end 37. In the illustrated embodiment, the interior groove 32 is spaced 0.325 inches from the open distal end 37; the illustrated groove 44 comprises a full radius of 0.200 inches. The illustrated second orthopaedic trial 11 may also have anti-rotation tabs at the distal end. It should be understood that the dimensions and particular shape and features of the illustrated second orthopaedic component are provided for purposes of illustration only; the invention is not intended to be limited to these dimensions, shape or features unless expressly called for in the claims.

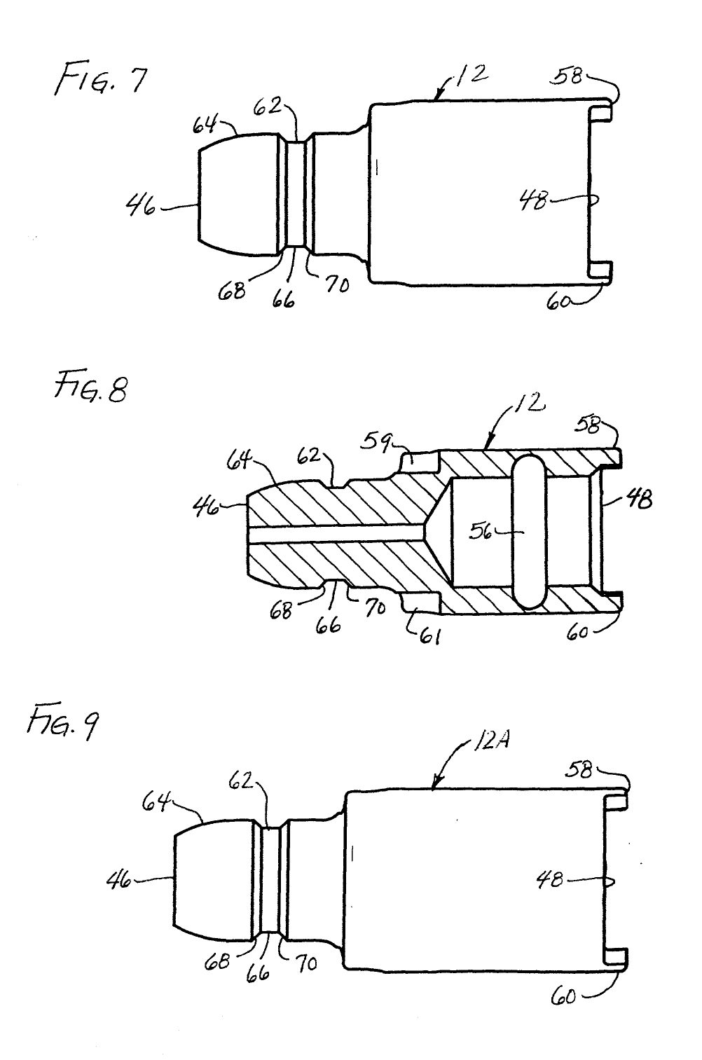

An embodiment of a third orthopaedic trial 12 is illustrated in FIGS. 7-8. The illustrated third orthopaedic trial 12 comprises a segmental component trial. It has an exterior shape that is similar to that of one of the modular segmental implant components that may ultimately by used, with male and female ends 46, 48 respectively. As shown in FIG. 8, the third orthopaedic trial 12 includes an interior wall 50 that defines a bore 52 with a longitudinal axis 54. As shown in FIG. 8, the interior wall 52 also has a circular interior groove 56 near the open female end 48. In the illustrated embodiment, the interior groove 56 is spaced 0.325 inches from the open female end 48; the illustrated interior groove 56 comprises a full radius of 0.200 inches. The illustrated third orthopaedic trial 12 also has anti-rotation tabs 58, 60 at the distal end, and slots 59, 61 between the two ends 46, 48. All of the trials may include complementary slots and anti-rotation tabs, so that the tabs fit within the slots and prevent relative rotation between assembled trials. It should be understood that the dimensions and particular shape and features of the illustrated second orthopaedic component are provided for purposes of illustration only; the invention is not intended to be limited to these dimensions, shape or features unless expressly called for in the claims.

The third orthopaedic trial 12 also has an exterior groove 62 near the male end 46. The exterior groove 62 is spaced from the male end 46, and the trial has a curved tapered exterior surface 64 that extends from the exterior groove 62 to the male end 46. In the illustrated embodiment, the exterior groove 62 is defined by a cylindrical surface 66 and two chamfered surfaces 68, 70 that diverge out from the cylindrical surface.

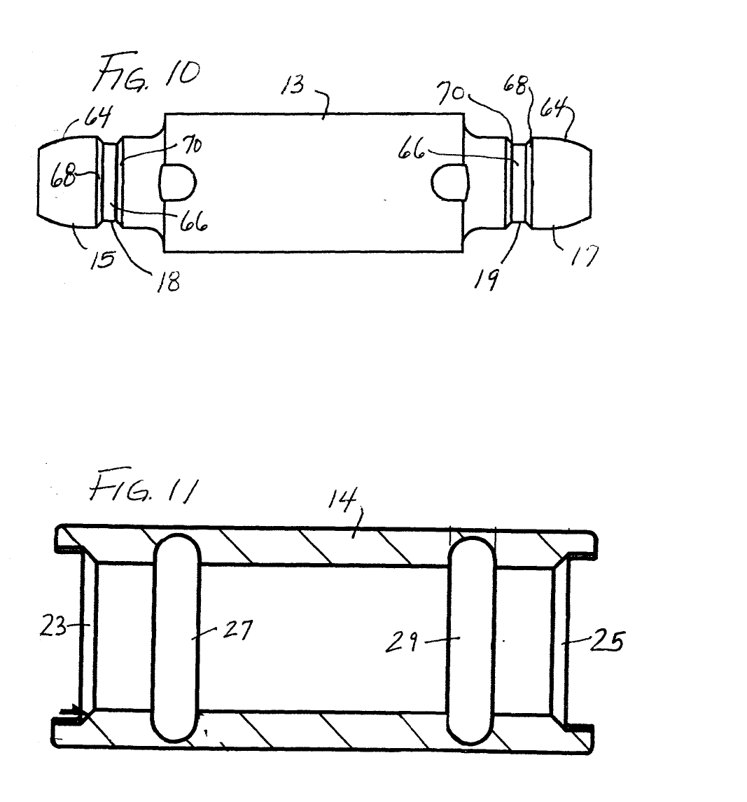

A surgical kit can also include segmental component trials with two male ends and two female ends. Examples are illustrated in FIGS. 10-11, respectively. In these embodiments, the segmental component trial 13 with two male ends 15, 17 includes two exterior grooves 18, 19 and the segmental component trial 14 with two female ends 23, 25 includes two interior grooves 27, 29. These exterior grooves 18, 19 may be shaped and dimensioned like those described above for the embodiments of FIGS. 7-9. These interior grooves 27, 29 may be shaped and dimensioned like those described above for the embodiments of FIGS. 1-9.

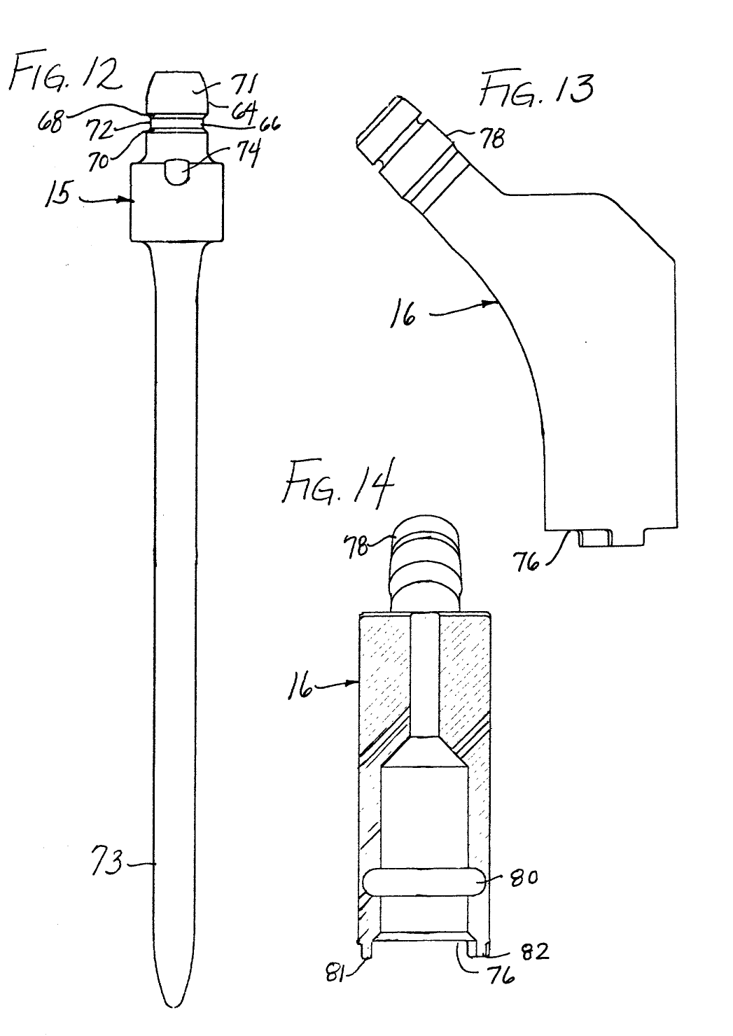

An embodiment of another orthopaedic trial 15 is illustrated in FIG. 12. The orthopaedic trial 15 of FIG. 12 comprises a femoral stem trial. It's exterior shape is similar to that of the modular femoral stem implant component that will ultimately by used. As shown in FIG. 12, the femoral stem trial 15 has a distal male end 71 with an exterior groove 72 similar in size and shape to the male ends 46, 15, 17 and exterior grooves 62, 18, 19 shown in FIGS. 7-11. The proximal end comprises a stem 73 shaped to be received in the intramedullary canal of the femur after reaming. The illustrated femoral stem trial 15 may also have anti-rotation slots 74 near the distal end to receive the anti-rotation tabs of another element. The distal male end 71 of the femoral stem trial may be received in the proximal female end 24 of the distal femoral trial 10 shown in FIGS. 1-3, or may be received, for example in the open female end 48 of one of the segmental component trials 12, 12A if the surgeon determines that extra length is necessary or desirable. Although not shown separately, it should be understood that a similar distal tibial stem trial may be provided, with a male end to be received in the open distal female end 37 of the tibial trial 11 of FIGS. 4-6.

An embodiment of another orthopaedic trial 16 is illustrated in FIGS. 13-14. The orthopaedic trial 16 of FIGS. 13-14 comprises a proximal femoral trial. It's exterior shape is similar to that of the modular proximal femoral implant component that may ultimately by used. As shown in FIG. 14, the proximal femoral trial 16 has a distal female end 76 and a proximal end 78 that is shaped to receive ball element trials (not shown). Like the other trials shown with open female ends, the proximal femoral trial 16 includes an interior groove 80. This interior groove 80 may be shaped and dimensioned like the above described interior grooves, e.g. 32, 44, 56. The distal female end 76 of the proximal femoral trial 16 may receive the male end of another trial, such as male element 46, 15, 17, 71. The illustrated proximal femoral trial 16 may also have anti-rotation tabs 81, 82 that mate with anti-rotation slots in one of the other modular trials.

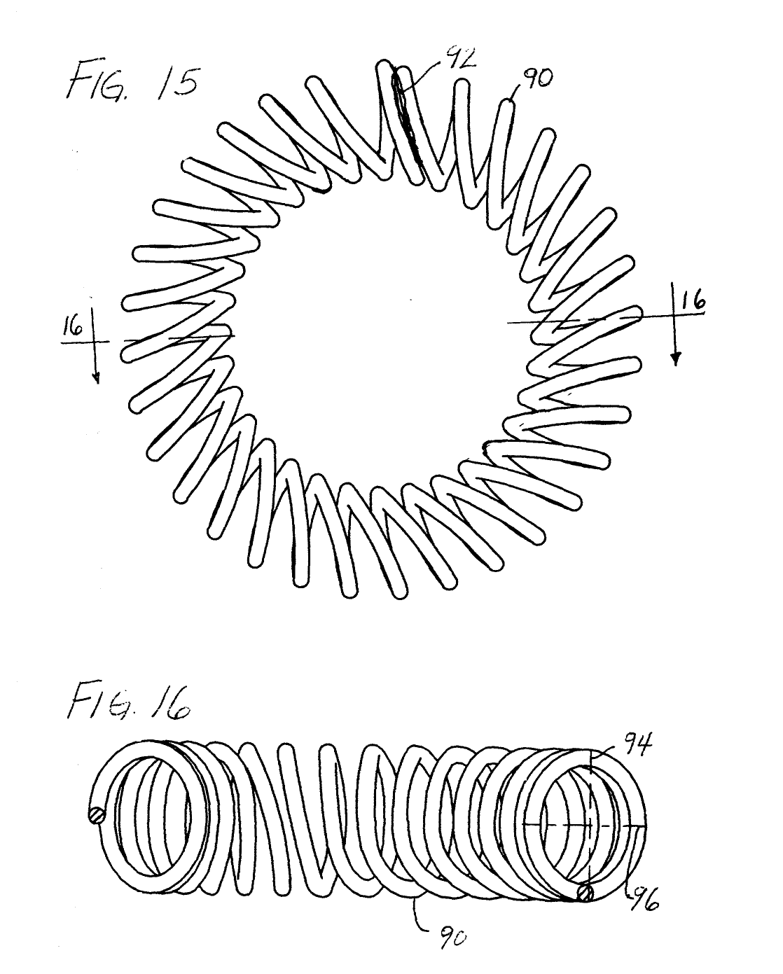

To allow the above-described components to be easily and quickly assembled and disassembled, a garter-type canted-coil spring 90 is provided for each interior groove 32, 44, 56, 80. An example of a garter-type canted-coil spring is illustrated in FIGS. 15-16. It is a commercial product purchased from Bal Seal Engineering Co., Inc. of Foothill Ranch, Calif. (Part No. X205498). It is a stainless steel spring with an inner diameter of 0.640 inches, with the two ends lap welded together, as shown at 92 in FIG. 15, to form the garter shape. The spring 90 has elliptical coils, with a major axis of 0.191 inches, shown at 94 in FIG. 16, and a minor axis of 0.162 inches, shown at 96 in FIG. 16. Similar springs may be used in each interior groove 32, 44, 56, 80. The springs 90 may be like those described in the following U.S. Patents, which are incorporated by reference herein in their entireties: U.S. Pat. Nos. 4,655,462; 4,826,144; 4,830,344; 4,876,781; 4,915,366; 4,934,666; 5,072,070; 5,079,388; 5,117,066; 5,082,390; 5,108,078; and U.S. Pat. No. 5,139,276, for example. This commercial product, these dimensions and these patents are identified for purposes of illustration only; the present invention is not intended to be limited to any particular product or dimension or characteristic unless expressly called for in the claims.

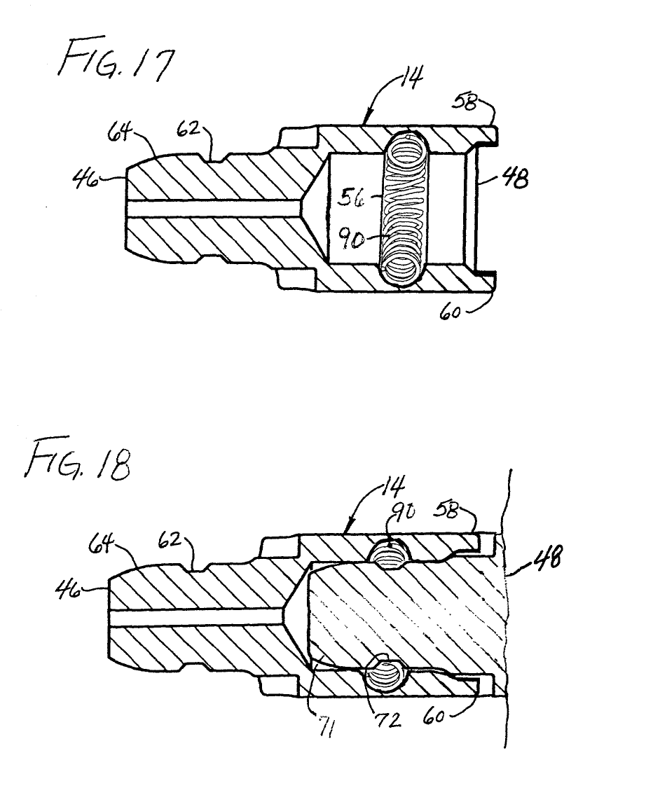

In the illustrated embodiments, one such canted-coil spring 90 is assembled with each trial 10, 11, 12, 14, 16 having an interior groove 32, 44, 56, 80. Such an assembly is illustrated in FIG. 17. Each interior groove 32, 44, 56, 80 is sized and shaped to receive a portion of one canted-coil spring 90. Each exterior groove 62, 18, 19, 72 is sized and shaped to receive a portion of one canted-coil spring 90.

A kit in accordance with the present invention may include several lengths and shapes of modular trials. For example, the segmental component trial 12A of FIG. 9 is substantially the same as that of FIGS. 7-8, except it is longer. Several lengths and shapes (e.g. bowed and straight) of modular femoral and tibial stem trials can be included in a single surgical kit, to match the sizes of modular implant elements that may be used. With several lengths of modular trials and modular implant elements, the surgeon should be able to select the best size and shape of implant for the particular patient's needs.

In use, when the surgeon is ready to being trialing, the surgeon may assemble modular component trials using, for example, the modular distal femoral trial 10, modular tibial trial 11, a plurality of modular segmental components 12, 12A, femoral stem trial 15 and tibial stem trial, and proximal femoral trial 16. Modular segmental components 13, 14 such as those shown in FIGS. 10-11 may also be used. To assemble any two of the modular components 10, 11, 12, 12A, 13, 14, 15, 16, the surgeon places the male end of one modular trial into the open female end of a second modular trial; for example, the surgeon may place the male end 71 of the femoral stem trial 15 into the open proximal female end 24 of the distal femoral component 10. As the male end, e.g. 71 is pushed into the opening, e.g. 24, the curved tapering exterior surface 64 of the male end 71 pushes against the inner diameter of the canted coil spring 90 that is within the groove, e.g. 32, compressing the spring 90 by canting it. When the exterior groove, e.g. 72, of the male end, e.g. 71 aligns with the spring 90, the spring 90 expands into the exterior groove 72 and temporarily locks the two trials together, limiting relative longitudinal movement between them. Such an assembly is illustrated in FIG. 18. The anti-rotation tabs and slot are also aligned to prevent relative rotation between the two trials. All of the modular trials can be assembled in various combinations in the same manner, giving the surgeon great flexibility in trialing, as well as providing ease of assembly.

Disassembly of two connected trials is similarly quick and easy. The surgeon must pull the two trials apart with sufficient force to cause relative motion between the spring 90 and the chamfered surface 68 of the male end 71, causing the spring to compress 90. The two trials can then be separated.

It should be understood that although particular shapes have been shown for the interior grooves 32, 44, 56, 80 and exterior grooves 62, 18, 19, 72, the invention is not intended to be limited to any particular shape or size of groove unless expressly set forth in the claims. For example, instead of full radii for the interior grooves, a plurality of straight segments could be used. Various groove shapes are illustrated in the above-mentioned patents related to the canted coil springs. It is expected that the relative shapes, sizes and properties of the canted coil springs and grooves may be varied as taught in those patents and other patents related to such springs and in commercial literature related to such springs.

It should also be understood that although in the illustrated embodiments the canted coil springs 90 are shown held in the interior grooves, it is possible that the canted coil springs could be held in the exterior grooves.

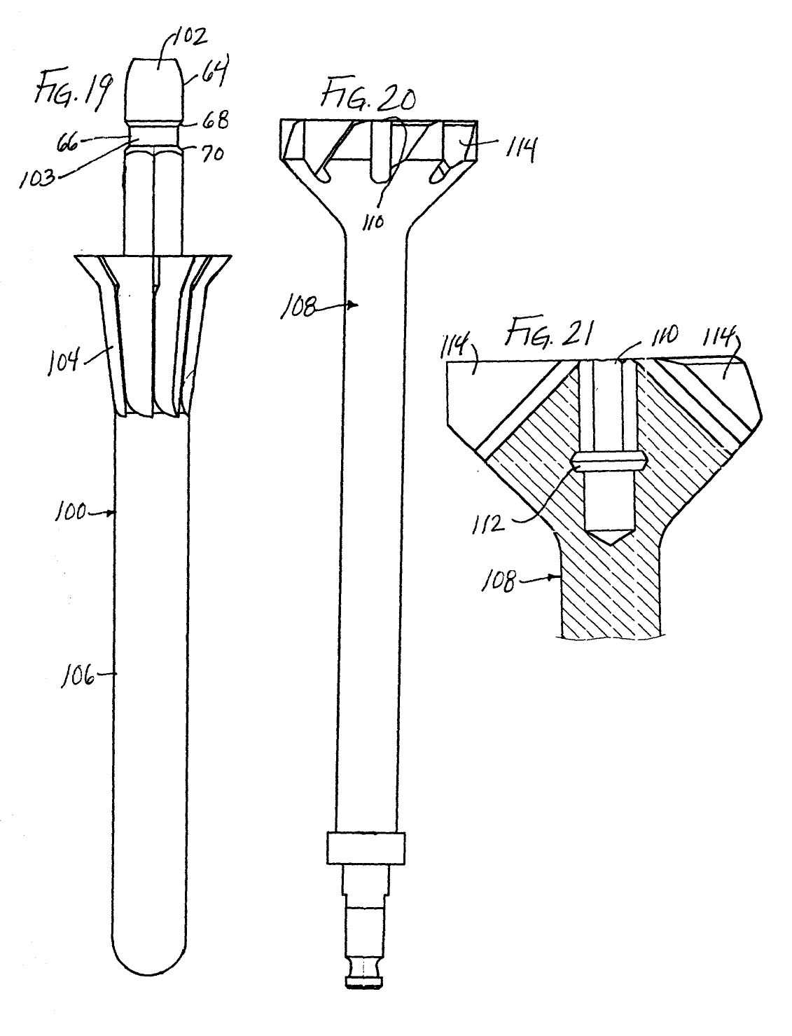

The principles of the present invention may also be applied to instruments that are used in surgery. Examples of instruments are illustrated in FIGS. 19-21. FIG. 19 illustrates a reamer 100 with a male end 102 that has an exterior groove 103 like those described above for the trials. The reamer 100 has cutting surfaces 104 and a stem portion 106. FIGS. 20-21 illustrate a Calcar Planar 108 that is used in conjunction with the reamer 100. The Calcar Planar 108 includes an open female end 110 and has an interior groove 112 similar to those described above for the trials. A garter-type canted coil spring like that shown in FIGS. 15-16 and described above is assembled with the Calcar Planar 108 such that the spring is carried in the interior groove 112. The open female end 110 of the planar 108 is surrounded by cutting surfaces 114. The two instruments can be assembled as described above for two trials, inserting the male end 102 into the open female end 110 until the garter-type canted coil spring is received in both the exterior groove 103 of the reamer 100 and the interior groove 112 of the planar 108. This design allows for modular tools: various sizes and shapes of reamers can be easily and quickly assembled with various sizes and shapes of planars, as well as easily and quickly disassembled to give the surgeon great flexibility.

It is anticipated that the principles of the present invention may be applied to other bones, such as in the arm. Generally, in any orthopaedic setting where modularity is desirable, in either instruments and trials, the principles of the present invention may find utility.

While only specific embodiments of the invention have been described and shown, it is apparent that various alternatives and modifications can be made thereto. Moreover, those skilled in the art will also recognize that certain additions can be made to these embodiments. It is, therefore, the intention in the appended claims to cover all such alternatives, modifications and additions as may fall within the true scope of the invention.

I claim:

1. A set of modular orthopaedic components comprising:
a first orthopaedic component having an interior groove;
a second orthopaedic component having an exterior groove;
a garter-type canted coil spring received in one of the grooves.

2. The set of modular components of claim 1 wherein the first and second modular components comprise modular orthopaedic trials.

3. The set of modular components of claim 1 wherein the first and second modular components comprise modular instruments.

4. The set of modular components of claim 1 wherein the garter-type canted coil spring is received in the interior groove.

5. An assembly of modular orthopaedic components comprising:
a first modular orthopaedic element having a bore and an interior groove;
a second modular orthopaedic element having a male portion with an exterior groove;
a garter-type canted coil spring;
wherein the male portion of the second modular orthopaedic element is received in the bore of the first modular orthopaedic element and wherein at least part of the garter-type canted coil spring is received within the interior groove of the first modular orthopaedic element and at least part of the garter-type canted coil spring is received with the exterior groove of the second modular orthopaedic element.

6. The assembly of claim 5 wherein the first and second modular orthopaedic elements comprise trials.

7. The assembly of claim 5 wherein the first and second modular orthopaedic elements comprise instruments.
